(19) United States
(12) Patent Application Publication
(10) Pub. No.: US 2019/0175786 A1
(43) Pub. Date: Jun. 13, 2019

(54) ELECTROSPUN FIBERS FOR THE REPAIR AND REGROWTH OF HYALINE CARTILAGE

(71) Applicants: Brian S. COHEN, Dublin, OH (US); Anthony A. ROMEO, Chicago, IL (US); Jed JOHNSON, London, OH (US); Devan OHST, Columbus, OH (US)

(72) Inventors: Brian S. COHEN, Dublin, OH (US); Anthony A. ROMEO, Chicago, IL (US); Jed JOHNSON, London, OH (US); Devan OHST, Columbus, OH (US)

(21) Appl. No.: 16/213,792

(22) Filed: Dec. 7, 2018

Related U.S. Application Data
(60) Provisional application No. 62/596,184, filed on Dec. 8, 2017.

(51) Int. Cl. A61L 27/26; A61L 27/54; A61L 27/12; A61L 27/10; A61L 27/36; A61L 24/10

(52) U.S. Cl. CPC A61L 27/26; A61L 27/54; A61L 27/12; A61L 27/10; A61L 27/3604; A61L 2400/12; A61L 24/106; A61L 24/104; A61L 24/102; A61L 2430/06; A61L 27/3608

(57) ABSTRACT

The instant disclosure is directed to methods of treating articular or hyaline cartilage damage or injury using biocompatible electrospun polymer fibers. Methods are also directed to treating arthritis, particularly osteoarthritis or rheumatoid arthritis, using biocompatible electrospun polymer fibers. Such methods may involve placing a patch comprising at least one electrospun polymer fiber in physical communication with the damaged cartilage. In certain embodiments, the patch may comprise substantially parallel electrospun polymer fibers.

### BACKGROUND

Articular cartilage is the smooth, white tissue that covers the ends of bones where they come together to form joints. The main component of the joint surface is a special tissue called hyaline cartilage. Hyaline cartilage is the glass-like (hyaline) but translucent cartilage that is found on many joint surfaces. It is pearl-grey in color with firm consistency and has a considerable amount of collagen. It contains no nerves or blood vessels, and its structure is relatively simple. Healthy cartilage in our joints makes it easier to move. It allows the bones to glide over each other with very little friction.

Articular cartilage can be damaged by injury or by normal wear and tear. When it is damaged, the joint surface may no longer be smooth. Moving bones along a damaged joint surface is difficult and may cause pain and/or swelling. Damaged cartilage can also lead to arthritis in the joint.

Because cartilage does not heal itself well, there exists a need for methods to restore, repair, and/or regrow articular cartilage. When the damage increases and the chondral defect reaches the subchondral bone, the blood supply in the bone starts a healing process in the defect. Scar tissue made up of a type of cartilage called fibrocartilage is then formed. Although fibrocartilage is able to fill in articular cartilage defects, its structure is significantly different from that of hyaline cartilage; it is much denser and it does not withstand the demands of everyday activities as well as hyaline cartilage. It is therefore at a higher risk of breaking down.

Restoring articular cartilage can relieve pain and allow better function. Restoring articular cartilage would also have the beneficial effects of slowing down the progression of damage or considerably delaying joint replacement (e.g. knee replacement) surgery. It is intended that the articular restoration methods of this disclosure will help patients return to their prior lifestyle, such as by regaining mobility, going back to work, and even engaging in sports again.

### SUMMARY

The instant disclosure is directed to methods of using electrospun fibers to repair and/or regrow hyaline cartilage. In some embodiments, a method of treating hyaline cartilage damage in a subject in need thereof may comprise placing at least one electrospun polymer fiber in physical communication with damaged hyaline cartilage of the subject. In certain embodiments, a method of treating arthritis in a subject in need thereof may comprise placing at least one electrospun polymer fiber in physical communication with hyaline cartilage of the subject.

### DESCRIPTION OF THE DRAWINGS

FIG. 1A illustrates repair of defects on the distal surface of a rabbit femur using Cartiform® Viable Osteochondral Allograft (CART) in which one side of a bilateral 4.75 mm diameter approximately 4 mm deep defect was repaired with Cartiform®.

FIG. 1B illustrates repair of defects on the distal surface of a rabbit femur using a nanofiber scaffold in which one side of a bilateral 4.75 mm diameter approximately 4 mm deep defect was repaired with a nanofiber scaffold in accordance with the instant disclosure.

FIG. 2A illustrates a histological sample of the femur repaired using Cartiform®.

FIG. 2B illustrates a histological sample of the femur repaired using the nanofiber scaffold in accordance with the instant disclosure.

FIG. 3A illustrates the nanofiber scaffold in accordance with the instant disclosure. at a magnification of 140×.

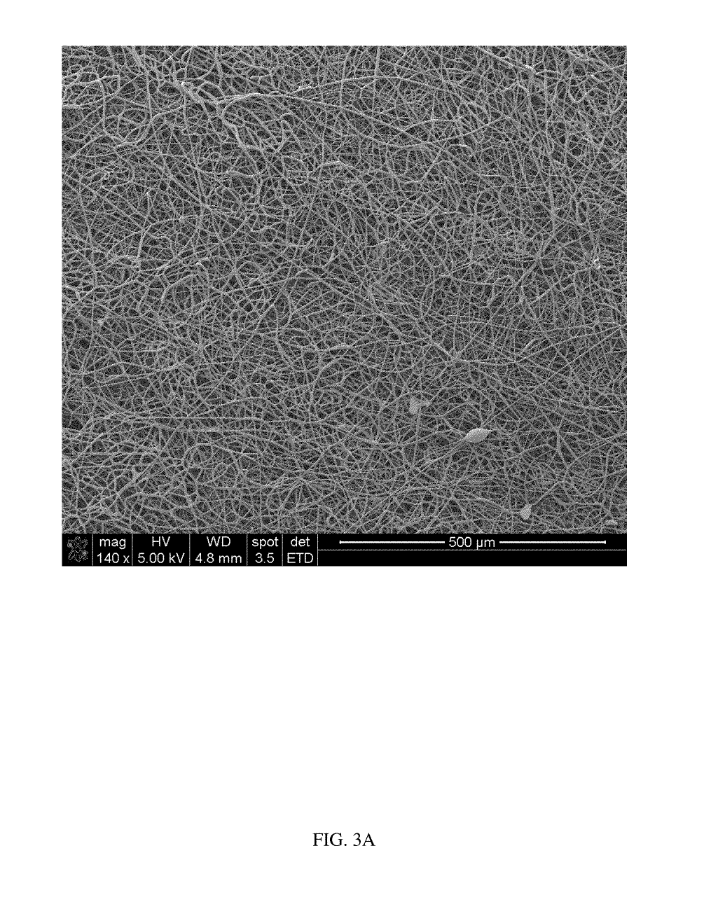

FIG. 3B illustrates the nanofiber scaffold in accordance with the instant disclosure. at a magnification of 1200×.

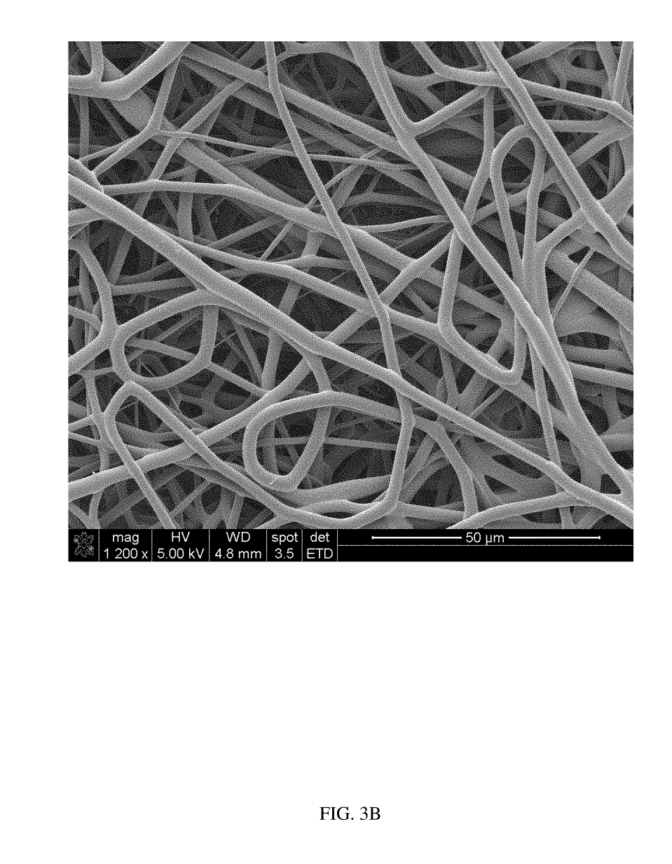

FIG. 3C illustrates the nanofiber scaffold in accordance with the instant disclosure. at a magnification of 5000×.

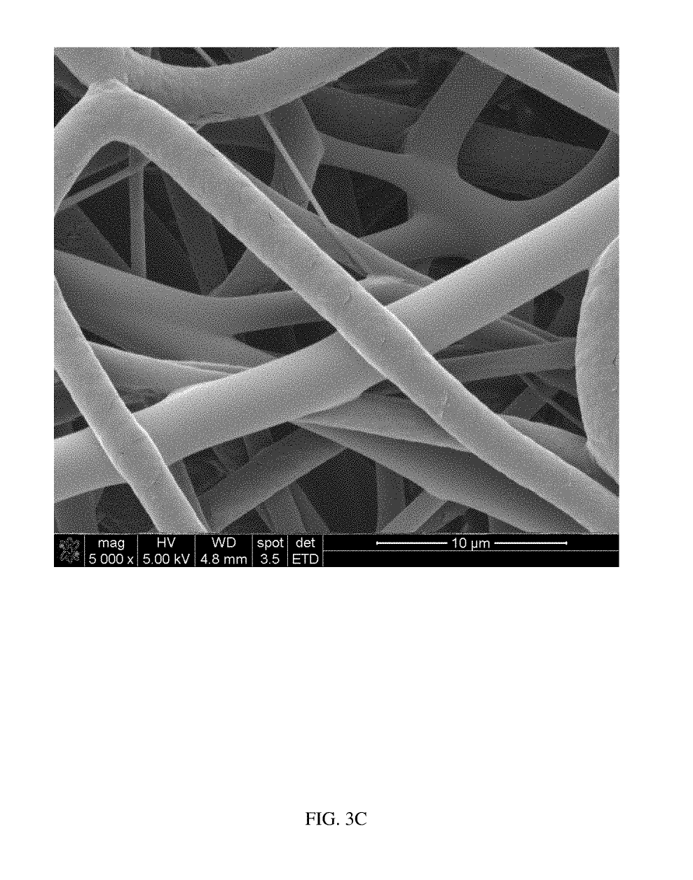

### DETAILED DESCRIPTION

Before the present compositions and methods are described, it is to be understood that this invention is not limited to the particular processes, formulations, compositions, or methodologies described, as these may vary. It is also to be understood that the terminology used in the description is for the purpose of describing the particular versions or embodiments only, and is not intended to limit the scope of embodiments herein which will be limited only by the appended claims. Unless defined otherwise, all technical and scientific terms used herein have the same meanings as commonly understood by one of ordinary skill in the art. Although any methods and materials similar or equivalent to those described herein can be used in the practice or testing of embodiments of embodiments herein, the preferred methods, devices, and materials are now described. All publications mentioned herein are incorporated by reference in their entirety. Nothing herein is to be construed as an admission that embodiments herein is not entitled to antedate such disclosure by virtue of prior invention.

The following terms shall have, for the purposes of this application, the respective meanings set forth below. Unless otherwise defined, all technical and scientific terms used herein have the same meanings as commonly understood by one of ordinary skill in the art. Nothing in this disclosure is to be construed as an admission that the embodiments described in this disclosure are not entitled to antedate such disclosure by virtue of prior invention.

As used herein, the singular forms “a,” “an,” and “the” include plural references, unless the context clearly dictates otherwise. Thus, for example, reference to a “fiber” is a reference to one or more fibers and equivalents thereof known to those skilled in the art, and so forth.

As used herein, the term “about” means plus or minus 10% of the numerical value of the number with which it is being used. Therefore, about 50 mm means in the range of 45 mm to 55 mm.

As used herein, the term “consists of” or “consisting of” means that the device or method includes only the elements, steps, or ingredients specifically recited in the particular claimed embodiment or claim.

In embodiments or claims where the term comprising is used as the transition phrase, such embodiments can also be envisioned with replacement of the term “comprising” with the terms “consisting of” or “consisting essentially of.”

The terms “animal,” “patient,” and “subject” as used herein include, but are not limited to, humans and non-human vertebrates such as wild, domestic and farm animals. In some embodiments, the terms “animal,” “patient,” and “subject” may refer to humans.

As used herein, the term “biocompatible” refers to non-harmful compatibility with living tissue. Biocompatibility is a broad term that describes a number of materials, including bioinert materials, bioactive materials, bioabsorbable materials, biostable materials, biotolerant materials, or any combination thereof.

As used herein, the term “improve” is used to convey that the methods of healing cartilage as described in embodiments herein change either the appearance, form, characteristics and/or the physical attributes of the tissue to which it is being provided, applied or administered.

The terms “heal,” “treat,” “treated,” or “treating,” as used herein, refers to both therapeutic treatment and prophylactic or preventative measures, wherein the object is to inhibit, prevent or slow down (lessen) an undesired physiological condition, disorder or disease, or to improve, inhibit, or otherwise obtain beneficial or desired clinical results. For the purposes of this invention, beneficial or desired clinical results include, but are not limited to, full or partial restoration of the cartilage tissue, full or partial regrowth of the cartilage tissue, full or partial repair of the cartilage tissue, prevention of further cartilage injury, improvement or alleviation of symptoms, including pain or swelling; diminishment of the extent of the condition, disorder or disease; stabilization (i.e., not worsening) of the state of the condition, disorder or disease; delay in onset or slowing of the progression of the condition, disorder or disease; amelioration of the condition, disorder or disease state; and remission (whether partial or total), whether detectable or undetectable, or enhancement or improvement of the condition, disorder or disease.

Hyaline cartilage is the name of the tough, flexible tissue that serves as a cushion for bones at joints, preventing them from rubbing against each other during physical activity. When hyaline cartilage is damaged as a result of trauma or gradual wear and tear normal movement of the joint can become limited and patients can experience severe pain as bones begin to grind against each other. Both can lead to disability over time.

Unfortunately, the body cannot readily repair damage to cartilage, because unlike most tissues in the body, cartilage does not have its own blood supply to bathe damaged tissue and provide factors promoting regeneration. Consequently, surgeons currently employ a technique called microfracture surgery to facilitate new cartilage growth. The surgery is generally performed in young adults who have a tear in the cartilage that surrounds the knee as a result of sports injury and is not effective in patients with widespread cartilage degeneration or osteoarthritis.

The instant disclosure is directed to methods of using electrospun fibers to repair and/or regrow hyaline cartilage. In some embodiments, a method of treating hyaline cartilage damage in a subject in need thereof may comprise placing at least one electrospun polymer fiber in physical communication with damaged hyaline cartilage of the subject. In certain embodiments, a method of treating arthritis in a subject in need thereof may comprise placing at least one electrospun polymer fiber in physical communication with hyaline cartilage of the subject.

In one embodiment, a method of healing hyaline cartilage damage may include placing a patch or scaffold comprising at least one electrospun polymer fiber in physical communication with the articular joint or the articular cartilage. In certain embodiments, the patch may comprise substantially parallel electrospun polymer fibers. In some embodiments, the electrospun fibers are aligned to mimic the native collagen architecture of the collagen fibers in cartilage.

### Electrospinning Fibers

Electrospinning is a method which may be used to process a polymer solution into a fiber. In embodiments wherein the diameter of the resulting fiber is on the nanometer scale, the fiber may be referred to as a nanofiber. Fibers may be formed into a variety of shapes by using a range of receiving surfaces, such as mandrels or collectors. In some embodiments, a flat shape, such as a sheet or sheet-like fiber mold, a fiber scaffold and/or tube, or a tubular lattice, may be formed by using a substantially round or cylindrical mandrel. In certain embodiments, the electrospun fibers may be cut and/or unrolled from the mandrel as a fiber mold to form the sheet. The resulting fiber molds or shapes may be used in many applications, including the repair or replacement of biological structures. In some embodiments, the resulting fiber scaffold may be implanted into a biological organism or a portion thereof.

Electrospinning methods may involve spinning a fiber from a polymer solution by applying a high DC voltage potential between a polymer injection system and a mandrel. In some embodiments, one or more charges may be applied to one or more components of an electrospinning system. In some embodiments, a charge may be applied to the mandrel, the polymer injection system, or combinations or portions thereof. Without wishing to be bound by theory, as the polymer solution is ejected from the polymer injection system, it is thought to be destabilized due to its exposure to a charge. The destabilized solution may then be attracted to a charged mandrel. As the destabilized solution moves from the polymer injection system to the mandrel, its solvents may evaporate and the polymer may stretch, leaving a long, thin fiber that is deposited onto the mandrel. The polymer solution may form a Taylor cone as it is ejected from the polymer injection system and exposed to a charge.

### Polymer Injection System

A polymer injection system may include any system configured to eject some amount of a polymer solution into an atmosphere to permit the flow of the polymer solution from the injection system to the mandrel. In some embodiments, the polymer injection system may deliver a continuous or linear stream with a controlled volumetric flow rate of a polymer solution to be formed into a fiber. In some embodiments, the polymer injection system may deliver a variable stream of a polymer solution to be formed into a fiber. In some embodiments, the polymer injection system may be configured to deliver intermittent streams of a polymer solution to be formed into multiple fibers. In some embodiments, the polymer injection system may include a syringe under manual or automated control. In some embodiments, the polymer injection system may include multiple syringes and multiple needles or needle-like components under individual or combined manual or automated control. In some embodiments, a multi-syringe polymer injection system may include multiple syringes and multiple needles or needle-like components, with each syringe containing the same polymer solution. In some embodiments, a multi-syringe polymer injection system may include multiple syringes and multiple needles or needle-like components, with each syringe containing a different polymer solution. In some embodiments, a charge may be applied to the polymer injection system, or to a portion thereof. In some embodiments, a charge may be applied to a needle or needle-like component of the polymer injection system.

In some embodiments, the polymer solution may be ejected from the polymer injection system at a flow rate of less than or equal to about 5 mL/h per needle. In other embodiments, the polymer solution may be ejected from the polymer injection system at a flow rate per needle in a range from about 0.01 mL/h to about 50 mL/h. The flow rate at which the polymer solution is ejected from the polymer injection system per needle may be, in some non-limiting examples, about 0.01 mL/h, about 0.05 mL/h, about 0.1 mL/h, about 0.5 mL/h, about 1 mL/h, about 2 mL/h, about 3 mL/h, about 4 mL/h, about 5 mL/h, about 6 mL/h, about 7 mL/h, about 8 mL/h, about 9 mL/h, about 10 mL/h, about 11 mL/h, about 12 mL/h, about 13 mL/h, about 14 mL/h, about 15 mL/h, about 16 mL/h, about 17 mL/h, about 18 mL/h, about 19 mL/h, about 20 mL/h, about 21 mL/h, about 22 mL/h, about 23 mL/h, about 24 mL/h, about 25 mL/h, about 26 mL/h, about 27 mL/h, about 28 mL/h, about 29 mL/h, about 30 mL/h, about 31 mL/h, about 32 mL/h, about 33 mL/h, about 34 mL/h, about 35 mL/h, about 36 mL/h, about 37 mL/h, about 38 mL/h, about 39 mL/h, about 40 mL/h, about 41 mL/h, about 42 mL/h, about 43 mL/h, about 44 mL/h, about 45 mL/h, about 46 mL/h, about 47 mL/h, about 48 mL/h, about 49 mL/h, about 50 mL/h, or any range between any two of these values, including endpoints.

As the polymer solution travels from the polymer injection system toward the mandrel, the diameter of the resulting fibers may be in the range of about 0.1 μm to about 10 μm. Some non-limiting examples of electrospun fiber diameters may include about 0.1 μm, about 0.2 μm, about 0.25 μm, about 0.5 μm, about 1 μm, about 2 μm, about 5 μm, about 10 μm, about 20 μm, or ranges between any two of these values, including endpoints. In some embodiments, the electrospun fiber diameter may be from about 0.25 μm to about 20 μm.

### Polymer Solution

In some embodiments, the polymer injection system may be filled with a polymer solution. In some embodiments, the polymer solution may comprise one or more polymers. In some embodiments, the polymer solution may be a fluid formed into a polymer liquid by the application of heat. A polymer solution may include, for example, non-resorbable polymers, resorbable polymers, natural polymers, or a combination thereof.

In some embodiments, the polymers may include, for example, polyethylene terephthalate, polyurethane, polyethylene, polyethylene oxide, polyester, polymethylmethacrylate, polyacrylonitrile, silicone, polycarbonate, polyether ketone ketone, polyether ether ketone, polyether imide, polyamide, polystyrene, polyether sulfone, polysulfone, polyvinyl acetate, polytetrafluoroethylene, polyvinylidene fluoride, polycaprolactone, polylactic acid, polyglycolic acid, polylactide-co-caprolactone, polydioxanone, Poly(3-hydroxybutyrate-co-3-hydroxyvalerate), trimethylene carbonate, polydiols, polyesters, collagen, gelatin, fibrin, fibronectin, albumin, hyaluronic acid, elastin, chitosan, alginate, silk, and combinations thereof.

It may be understood that polymer solutions may also include a combination of one or more of non-resorbable, resorbable polymers, and naturally occurring polymers in any combination or compositional ratio. In an alternative embodiment, the polymer solutions may include a combination of two or more non-resorbable polymers, two or more resorbable polymers or two or more naturally occurring polymers. In some non-limiting examples, the polymer solution may comprise a weight percent ratio of, for example, from about 5% to about 90%. Non-limiting examples of such weight percent ratios may include about 5%, about 10%, about 15%, about 20%, about 25%, about 30%, about 33%, about 35%, about 40%, about 45%, about 50%, about 55%, about 60%, about 66%, about 70%, about 75%, about 80%, about 85%, about 90%, or ranges between any two of these values, including endpoints.

In some embodiments, the polymer solution may comprise one or more solvents. In some embodiments, the solvent may comprise, for example, acetone, dimethylformamide, dimethylsulfoxide, N-methylpyrrolidone, N,N-dimethylformamide, Nacetonitrile, hexanes, ether, dioxane, ethyl acetate, pyridine, toluene, xylene, tetrahydrofuran, trifluoroacetic acid, hexafluoroisopropanol, acetic acid, dimethylacetamide, chloroform, dichloromethane, water, alcohols, ionic compounds, or combinations thereof. The concentration range of polymer or polymers in solvent or solvents may be, without limitation, from about 1 wt % to about 50 wt %. Some non-limiting examples of polymer concentration in solution may include about 1 wt %, 3 wt %, 5 wt %, about 10 wt %, about 15 wt %, about 20 wt %, about 25 wt %, about 30 wt %, about 35 wt %, about 40 wt %, about 45 wt %, about 50 wt %, or ranges between any two of these values, including endpoints.

In some embodiments, the polymer solution may also include additional materials. Non-limiting examples of such additional materials may include radiation opaque materials, contrast agents, electrically conductive materials, fluorescent materials, luminescent materials, antibiotics, growth factors, vitamins, cytokines, steroids, anti-inflammatory drugs, small molecules, sugars, salts, peptides, proteins, cell factors, DNA, RNA, other materials to aid in non-invasive imaging, or any combination thereof. In some embodiments, the radiation opaque materials may include, for example, barium, tantalum, tungsten, iodine, gadolinium, gold, platinum, bismuth, or bismuth (III) oxide. In some embodiments, the electrically conductive materials may include, for example, gold, silver, iron, or polyaniline.

In some embodiments, the additional materials may be present in the polymer solution in an amount from about 1 wt % to about 1500 wt % of the polymer mass. In some non-limiting examples, the additional materials may be present in the polymer solution in an amount of about 1 wt %, about 5 wt %, about 10 wt %, about 15 wt %, about 20 wt %, about 25 wt %, about 30 wt %, about 35 wt %, about 40 wt %, about 45 wt %, about 50 wt %, about 55 wt %, about 60 wt %, about 65 wt %, about 70 wt %, about 75 wt %, about 80 wt %, about 85 wt %, about 90 wt %, about 95 wt %, about 100 wt %, about 125 wt %, about 150 wt %, about 175 wt %, about 200 wt %, about 225 wt %, about 250 wt %, about 275 wt %, about 300 wt %, about 325 wt %, about 350 wt %, about 375 wt %, about 400 wt %, about 425 wt %, about 450 wt %, about 475 wt %, about 500 wt %, about 525 wt %, about 550 wt %, about 575 wt %, about 600 wt %, about 625 wt %, about 650 wt %, about 675 wt %, about 700 wt %, about 725 wt %, about 750 wt %, about 775 wt %, about 800 wt %, about 825 wt %, about 850 wt %, about 875 wt %, about 900 wt %, about 925 wt %, about 950 wt %, about 975 wt %, about 1000 wt %, about 1025 wt %, about 1050 wt %, about 1075 wt %, about 1100 wt %, about 1125 wt %, about 1150 wt %, about 1175 wt %, about 1200 wt %, about 1225 wt %, about 1250 wt %, about 1275 wt %, about 1300 wt %, about 1325 wt %, about 1350 wt %, about 1375 wt %, about 1400 wt %, about 1425 wt %, about 1450 wt %, about 1475 wt %, about 1500 wt %, or any range between any of these two values, including endpoints.

The type of polymer in the polymer solution may determine the characteristics of the electrospun fiber. Some fibers may be composed of polymers that are bio-stable and not absorbable or biodegradable when implanted. Such fibers may remain generally chemically unchanged for the length of time in which they remain implanted. Alternatively, fibers may be composed of polymers that may be absorbed or bio-degraded over time. Such fibers may act as an initial template or scaffold during a healing process. These templates or scaffolds may degrade in vivo once the tissues have a degree of healing by natural structures and cells. It may be further understood that a polymer solution and its resulting electrospun fiber(s) may be composed or more than one type of polymer, and that each polymer therein may have a specific characteristic, such as bio-stability or biodegradability.

### Applying Charges to Electrospinning Components

In an electrospinning system, one or more charges may be applied to one or more components, or portions of components, such as, for example, a mandrel or a polymer injection system, or portions thereof. In some embodiments, a positive charge may be applied to the polymer injection system, or portions thereof. In some embodiments, a negative charge may be applied to the polymer injection system, or portions thereof. In some embodiments, the polymer injection system, or portions thereof, may be grounded. In some embodiments, a positive charge may be applied to mandrel, or portions thereof. In some embodiments, a negative charge may be applied to the mandrel, or portions thereof. In some embodiments, the mandrel, or portions thereof, may be grounded. In some embodiments, one or more components or portions thereof may receive the same charge. In some embodiments, one or more components, or portions thereof, may receive one or more different charges.

The charge applied to any component of the electrospinning system, or portions thereof, may be from about −15 kV to about 30 kV, including endpoints. In some non-limiting examples, the charge applied to any component of the electrospinning system, or portions thereof, may be about −15 kV, about −10 kV, about −5 kV, about −4 kV, about −3 kV, about −1 kV, about −0.01 kV, about 0.01 kV, about 1 kV, about 5 kV, about 10 kV, about 11 kV, about 11.1 kV, about 12 kV, about 15 kV, about 20 kV, about 25 kV, about 30 kV, or any range between any two of these values, including endpoints. In some embodiments, any component of the electrospinning system, or portions thereof, may be grounded.

### Mandrel Movement During Electrospinning

During electrospinning, in some embodiments, the mandrel may move with respect to the polymer injection system. In some embodiments, the polymer injection system may move with respect to the mandrel. The movement of one electrospinning component with respect to another electrospinning component may be, for example, substantially rotational, substantially translational, or any combination thereof. In some embodiments, one or more components of the electrospinning system may move under manual control. In some embodiments, one or more components of the electrospinning system may move under automated control. In some embodiments, the mandrel may be in contact with or mounted upon a support structure that may be moved using one or more motors or motion control systems. The pattern of the electrospun fiber deposited on the mandrel may depend upon the one or more motions of the mandrel with respect to the polymer injection system. In some embodiments, the mandrel surface may be configured to rotate about its long axis. In one non-limiting example, a mandrel having a rotation rate about its long axis that is faster than a translation rate along a linear axis, may result in a nearly helical deposition of an electrospun fiber, forming windings about the mandrel. In another example, a mandrel having a translation rate along a linear axis that is faster than a rotation rate about a rotational axis, may result in a roughly linear deposition of an electrospun fiber along a liner extent of the mandrel.

### Methods of Healing Cartilage Tissue Damage

The instant disclosure is directed to methods of using electrospun fibers to repair and/or regrow hyaline cartilage. It may be understood that the methods described herein may be applied to any articular cartilage tissue damage, and that the examples described herein are non-limiting.

Hyaline cartilage exists on the ventral ends of ribs; in the larynx, trachea, and bronchi; and on the articulating surfaces of bones. Some embodiments are directed to a method of treating hyaline cartilage damage in a subject in need thereof comprising administering a scaffold comprising one or more electrospun polymer fibers as described herein. In some embodiments, the scaffold may be a patch. In some embodiments, the electrospun fibers are aligned to mimic the native collagen architecture of the collagen fibers in cartilage. Collagen fibers in cartilage originate in the bone and are perpendicular to the articulating surface in the deep zone, and then transition to becoming parallel to the articulating surface in superficial layers. In some embodiments, the scaffold is adhered to the damaged hyaline cartilage. In some embodiments, the scaffold may be adhered to the damaged hyaline cartilage using sutures or a biological adhesive. In some embodiments, administering comprises placing the scaffold in physical connection with the cartilage. In some embodiments, administering comprises aligning the electrospun fibers to mimic the native collagen architecture of the collagen fibers in cartilage.

Articular cartilage has a limited capacity for intrinsic healing and repair. Some embodiments are directed to a method of treating articular cartilage damage in a subject in need thereof comprising administering a scaffold comprising one or more electrospun polymer fibers as described herein. In some embodiments, the articular cartilage may be hyaline cartilage. In some embodiments, the scaffold may be a plug, filling the void caused by the damage. In some embodiments, the scaffold may be a patch. Without wishing to be bound by theory, the scaffold may allow healing of the articular cartilage by providing a matrix for the cells to attach, or perhaps by facilitating the migration of cells from healthy tissues to the repair site.

In certain embodiments, aligning the electrospun polymer fibers to mimic the native collagen fiber alignment may further facilitate such migration and repair. In some embodiments, the scaffold is adhered to the damaged articular cartilage. In some embodiments, the scaffold may be adhered to the damaged articular cartilage using sutures or a biological adhesive.

In some embodiments, the method of treating articular cartilage damage using the scaffold comprising one or more electrospun polymer fibers does not require cell seeding. In some embodiments, the method of treating articular cartilage damage of embodiments herein does not require the administration of any biologics.

Some embodiments herein are also directed to methods of treating arthritis. In some embodiments, the arthritis may be primary or secondary osteoarthritis, or rheumatoid arthritis. In certain embodiments, a method of treating arthritis in a subject in need thereof comprises administering to an arthritic site of the subject a scaffold comprising one or more electrospun polymer fibers as described herein. In some embodiments, the scaffold is adhered to the damaged articular cartilage. In some embodiments, the scaffold may be adhered to the damaged articular cartilage using sutures or a biological adhesive. In some embodiments, the scaffold may be a plug, filling a void caused by the damage. In some embodiments, the scaffold may be a patch, layered over and into the damage.

In some embodiments, the suture may comprise one or more sutures. In some embodiments, the one or more sutures may extend through the scaffold. In another embodiment, the one or more sutures may extend through an opening in the scaffold. In some embodiments, the scaffold may surround or substantially surround the area from which the one or more sutures extends. In another embodiment, the one or more sutures may extend from approximately the center of the scaffold.

In some embodiments, the biological adhesive may be selected from the group consisting of fibrin sealants, autologous fibrin sealants, gelatin-resorcinol aldehydes, protein-aldehyde systems, collagen-based adhesives, polysaccharide-based adhesives, mussel adhesive proteins, and various biologically inspired or biomimetic glues as well as variants.

In some embodiments, the scaffold may be in physical communication with the damaged articular cartilage. In certain embodiments, the scaffold may comprise substantially parallel electrospun polymer fibers. In some embodiments, In some embodiments, the electrospun fibers are aligned to mimic the native collagen fiber alignment. In other embodiments, the scaffold may comprise randomly oriented electrospun polymer fibers. In still other embodiments, the scaffold may comprise a combination of randomly oriented and substantially parallel electrospun polymer fibers to mimic the native collagen architecture of the collagen fibers in cartilage. In still other embodiments, the scaffold may comprise a combination of substantially perpendicular, randomly oriented and substantially parallel electrospun polymer fibers to mimic the native collagen architecture of the collagen fibers in cartilage. Wicking may be used for the blood and bone marrow coming from the inferior part of the electrospun scaffold to help repair the defect.

In some embodiments, the scaffold meets one or more of the following requirements: biocompatible; biodegradable; highly porous; suitable for cell attachment, proliferation and differentiation; osteoconductive; noncytotoxic; flexible; elastic; and nonantigenic. In some embodiments, in order to improve cell attachment and growth, the electrospun polymer fibers may be subject to one or more of the following: radio frequency plasma, direct current-(DC-) pulsed oxygen plasma treatment, acrylic acid grafting, and collagen coating by covalent binding of collagen to carboxylic moieties of the polyacrylic acid.

In some embodiments, the patch may comprise one or more electrospun polymer fibers. In certain embodiments, the electrospun polymer fibers may have a diameter in the range of about 0.1 μm to about 10 μm. Some non-limiting examples of electrospun fiber diameters may include about 0.1 μm, about 0.2 μm, about 0.25 μm, about 0.5 μm, about 1 μm, about 2 μm, about 3 μm, about 4 μm, about 5 μm, about 6 μm, about 7 μm, about 8 μm, about 9 μm, about 10 μm, about 11 μm, about 12 μm, about 13 μm, about 14 μm, about 15 μm, about 16 μm, about 17 μm, about 18 μm, about 19 μm, about 20 μm, or ranges between any two of these values, including endpoints. In some embodiments, the electrospun fiber diameter may be from about 0.25 μm to about 20 μm.

In some embodiments, the patch may have, independently, a length from about 1 mm to about 100 mm, and a width from about 1 mm to about 100 mm. The patch may have, independently, a length or width of about, for example, about 1 mm, about 5 mm, about 10 mm, about 15 mm, about 20 mm, about 25 mm, about 30 mm, about 35 mm, about 40 mm, about 45 mm, about 50 mm, about 55 mm, about 60 mm, about 65 mm, about 70 mm, about 75 mm, about 80 mm, about 85 mm, about 90 mm, about 95 mm, about 100 mm, or any range between any two of these values, including endpoints.

In some embodiments, the patch may have a thickness from about 100 μm to about 5,000 m. The patch may have a thickness of, for example, about 100 m, about 200 m, about 300 m, about 400 m, about 500 m, about 600 m, about 700 m, about 800 m, about 900 m, about 1,000 m, about 1,250 m, about 1,500 m, about 1,750 m, about 2,000 m, about 2,250 m, about 2,500 m, about 2,750 m, about 3,000 m, about 3,250 m, about 3,500 m, about 3,750 m, about 4,000 m, about 4,250 m, about 4,500 m, about 4,750 m, about 5,000 μm, or any range between any two of these values, including endpoints.

In some embodiments, the patch may comprise one or more pores. In certain embodiments, the pores are uniformly, or substantially uniformly, distributed throughout the patch, while in other embodiments the pores are irregularly distributed within the patch. In some embodiments, the pores may have a diameter from about 0.25 μm to about 50 μm. The diameter of the pores may be, for example, about 0.25 m, about 0.5 μm, about 1 μm, about 2 μm, about 3 μm, about 4 μm, about 5 μm, about 10 μm, about 15 μm, about 20 μm, about 25 μm, about 30 μm, about 35 μm, about 40 μm, about 45 μm, about 50 μm, or any range between any two of these values, including endpoints.

In some embodiments, the patch may have particular mechanical properties, such as a particular Young's modulus, suture retention strength, radial stiffness, or bursting strength. In some embodiments, the Young's modulus of the patch may be from about 0.5 MPa to about 1,000 MPa. The Young's modulus may be, for example, about 0.5 MPA, about 1 MPa, about 10 MPa, about 20 MPa, about 30 MPa, about 40 MPa, about 50 MPa, about 60 MPa, about 70 MPa, about 80 MPa, about 90 MPa, about 100 MPa, about 110 MPa, about 120 MPa, about 130 MPa, about 140 MPa, about 150 MPa, about 160 MPa, about 170 MPa, about 180 MPa, about 190 MPa, about 200 MPa, about 210 MPa, about 220 MPa, about 230 MPa, about 240 MPa, about 250 MPa, about 260 MPa, about 270 MPa, about 280 MPa, about 290 MPa, about 300 MPa, about 310 MPa, about 320 MPa, about 330 MPa, about 340 MPa, about 350 MPa, about 360 MPa, about 370 MPa, about 380 MPa, about 390 MPa, about 400 MPa, about 410 MPa, about 420 MPa, about 430 MPa, about 440 MPa, about 450 MPa, about 460 MPa, about 470 MPa, about 480 MPa, about 490 MPa, about 500 MPa, about 510 MPa, about 520 MPa, about 530 MPa, about 540 MPa, about 550 MPa, about 560 MPa, about 570 MPa, about 580 MPa, about 590 MPa, about 600 MPa, about 610 MPa, about 620 MPa, about 630 MPa, about 640 MPa, about 650 MPa, about 660 MPa, about 670 MPa, about 680 MPa, about 690 MPa, about 700 MPa, about 710 MPa, about 720 MPa, about 730 MPa, about 740 MPa, about 750 MPa, about 760 MPa, about 770 MPa, about 780 MPa, about 790 MPa, about 800 MPa, about 810 MPa, about 820 MPa, about 830 MPa, about 840 MPa, about 850 MPa, about 860 MPa, about 870 MPa, about 880 MPa, about 890 MPa, about 900 MPa, about 910 MPa, about 920 MPa, about 930 MPa, about 940 MPa, about 950 MPa, about 960 MPa, about 970 MPa, about 980 MPa, about 990 MPa, about 1,000 MPa, or any range between any two of these values, including endpoints.

In some embodiments, the patch may comprise at least one layer of electrospun polymer fibers that are substantially parallel with respect to one another. In some embodiments, the patch may comprise more than one layer, and the electrospun polymer fibers of a first layer may be substantially parallel with respect to one another and substantially parallel to the fibers of any additional layers. In other embodiments, the patch may comprise more than one layer, and the electrospun polymer fibers of a first layer may be substantially parallel with respect to one another and substantially perpendicular to the fibers of any additional layers. A layer may include a sheet, such that a first layer may comprise a first sheet, and a second layer may comprise a second sheet, and so on, similar to how textiles may include more than one layer of material.

In some embodiments, the scaffold may be placed such that the electrospun polymer fibers mimic the native collagen architecture of the collagen fibers in cartilage. In some embodiments, the alignment of the electrospun polymer fibers may be configured to facilitate the migration of cells to the site of the repair.

In some embodiments, the patch may further comprise additional materials. The additional materials may be, for example, tricalcium phosphate, hydroxyapatite, bioglass, or any combination thereof.

In additional embodiments, the patch may further comprise a biologic component. The biologic component may be, for example, mesenchymal stem cells, tenocytes, fibroblasts, osteoblasts, platelet-rich plasma, stromal vascular fraction, bursa cells, amnion, growth factors, or any combination thereof.

In some embodiments, the at least one electrospun polymer fiber may comprise a polymer selected from the group consisting of polyethylene terephthalate, polyurethane, polyethylene, polyethylene oxide, polyester, polymethylmethacrylate, polyacrylonitrile, silicone, polycarbonate, polyether ketone ketone, polyether ether ketone, polyether imide, polyamide, polystyrene, polyether sulfone, polysulfone, polyvinyl acetate, polytetrafluoroethylene, polyvinylidene fluoride, polycaprolactone, polylactic acid, polyglycolic acid, polylactide-co-glycolide), polydioxanone, polylactide-co-caprolactone, polydioxanone, Poly(3-hydroxybutyrate-co-3-hydroxyvalerate), trimethylene carbonate, polydiols, polyesters, collagen, gelatin, fibrin, fibronectin, albumin, hyaluronic acid, elastin, chitosan, alginate, silk, any copolymer thereof, or any combination thereof. In some embodiments, the at least one electrospun polymer fiber may comprise about 20 wt % polyethylene terephthalate and about 80 wt % polyurethane. In other embodiments, the at least one electrospun polymer fiber may comprise a combination of polylactide-co-caprolactone and polyglycolic acid. In still other embodiments, the at least one electrospun polymer fiber may comprise at least a first fiber comprising polylactide-co-caprolactone fiber and at least a second fiber comprising polyglycolic acid fiber, wherein the first fiber and the second fiber are co-spun.

In another embodiment, the at least one electrospun polymer fiber has a diameter of about 0.25 μm to about 20 μm. In another embodiment, the patch has a length of about 1 mm to about 100 mm and a width of about 1 mm to about 100 mm. In another embodiment, the patch has a thickness of about 100 μm to about 5,000 μm. In another embodiment, the patch further comprises pores with a diameter of about 0.25 μm to about 50 μm. In another embodiment, the patch has a Young's modulus of about 0.5 MPa to about 500 MPa. In another embodiment, the patch comprises at least one layer of electrospun polymer fibers that are substantially parallel with respect to one another. In another embodiment, the patch is placed such that the substantially parallel electrospun polymer fibers are aligned to mimic the native collagen architecture of the collagen fibers in cartilage. In another embodiment, the substantially parallel electrospun polymer fibers are configured to facilitate the migration of cells. In another embodiment, the patch further comprises a material selected from the group consisting of tricalcium phosphate, hydroxyapatite, bioglass, and combinations thereof. In another embodiment, the patch further comprises a biologic component. In another embodiment, the biologic component is selected from the group consisting of mesenchymal stem cells, tenocytes, fibroblasts, osteoblasts, platelet-rich plasma, stromal vascular fraction, bursa cells, amnion, growth factors, and combinations thereof. In another embodiment, the at least one electrospun polymer fiber comprises a polymer selected from the group consisting of polyethylene terephthalate, polyurethane, polyethylene, polyethylene oxide, polyester, polymethylmethacrylate, polyacrylonitrile, silicone, polycarbonate, polyether ketone ketone, polyether ether ketone, polyether imide, polyamide, polystyrene, polyether sulfone, polysulfone, polyvinyl acetate, polytetrafluoroethylene, polyvinylidene fluoride, polycaprolactone, polylactic acid, polyglycolic acid, polylactide-co-caprolactone, polydioxanone, Poly(3-hydroxybutyrate-co-3-hydroxyvalerate), trimethylene carbonate, polydiols, polyesters, collagen, gelatin, fibrin, fibronectin, albumin, hyaluronic acid, elastin, chitosan, alginate, silk, and combinations thereof. In another embodiment, the at least one electro spun polymer fiber comprises about 20 wt % polyethylene terephthalate and about 80 wt % polyurethane. In another embodiment, the at least one electrospun fiber comprises a combination of polylactide-co-caprolactone and polyglycolic acid.

The embodiments illustrating the method and materials used may be further understood by reference to the following non-limiting example.

### Example 1

### Objectives

To characterize bone ingrowth and local tissues response of a cartilage repair with a novel, co-electrospun PLCL+PGA scaffold from Nanofiber Solutions.

### Model Type and Sacrifice Time Points:

### Test Devices

1. Cartiform® Viable Osteochondral Allograft (CART) was utilized as commercially available.
2. Nanofiber scaffold (NF) as provided by Nanofiber Solutions.

All nanofiber scaffolds were provided by Nanofiber Solutions. The Nanofiber Solutions scaffold was cut into a 4.75 mm diameter disc to match the defect size prior to implant/mixing with other substrates for implant.

### Experimental Design and Procedures

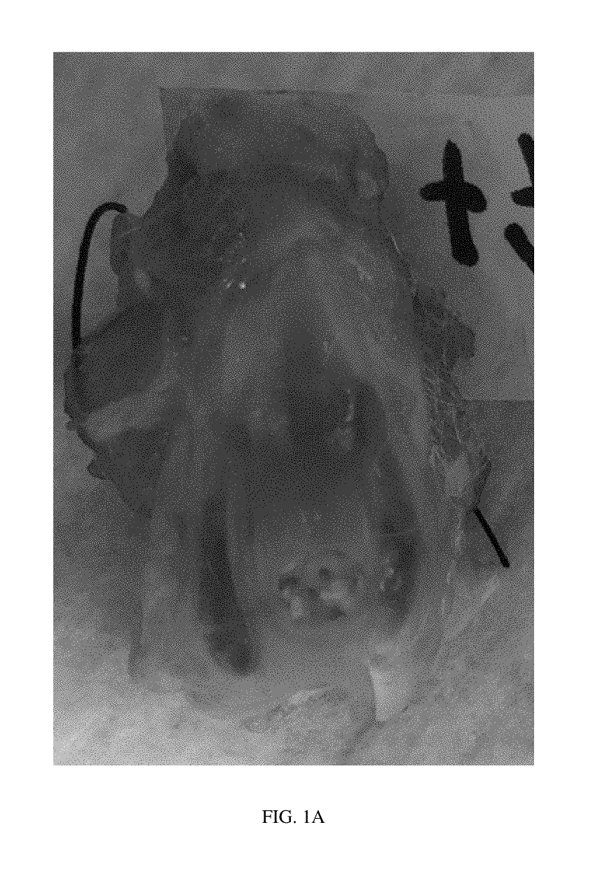

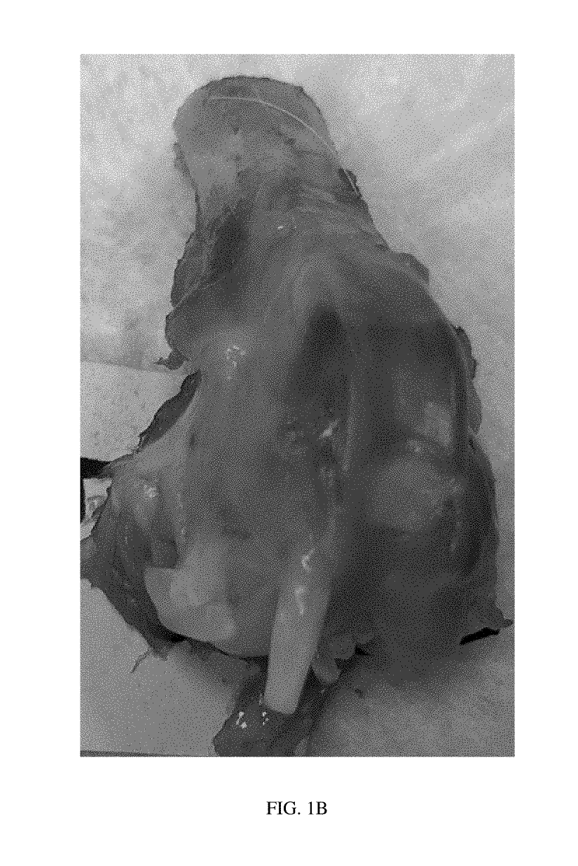

Each rabbit was anesthetized and underwent creation of two defects on the distal surface of the femurs. Briefly, lateral parapatellar skin incisions were made and the patellas were dislocated laterally. Afterwards, the knees were flexed to expose the medial femoral condyles. A defect was created (approximately 4.75 mm in diameter and 4 mm deep) extending through the cartilage into the subchondral bone. Following creation of the defect, each defect was assigned to receive one of two possible treatments:
1. Cartiform (See FIG. 1A)
2. Nanofiber scaffold (See FIG. 1B)

Following creation and repair of the defect, the rabbit was allowed to recover for up to 6 weeks. Following recovery, rabbits were euthanized and graft tissues harvested for histopathology assessment.

### Histology

Six rabbits (N=12 defects) were allocated to histology. At necropsy, gross dissection (i.e., limbs isolated, skin removed) were completed, following which, samples were placed in 10% neutral buffered formalin.

Following gross dissection and fixation (a minimum of two (N=2) weeks of in formalin) surgical defect sites were isolated by creating a slab of tissue approximately 1 cm think in the sagittal plane. Images were taken of the gross tissue blocks. After fixation, the tissue was dehydrated in graded solutions of ETOH on a tissue processor (Tissue-Tek VIP, Sakura, Torrance, Calif.). After processing, the samples were cleared with acetone and polymerized into a hardened plastic block using Hard Acrylosin (Dorn and Hart Microedge).

Histological sections were taken in the sagittal plane to display the defect site, articulating surface, and surrounding bone. One (N=1) slide was cut through each ROI. Initial sections were taken using an Exakt diamond blade bone saw at a thickness of approximately 300-400 μm. All sections were ground using an Exakt microgrinder to 60-70 μm thickness and stained. Sections were first stained with Sanderson's Rapid Bone stain, which provides differentiation of cells within the section and allows detection of cartilage within the tissue. Slides were then counterstained using a Van Gieson bone stain that allows differentiation of collagen and detection of bone (immature woven bone and mature lamellar bone) within the section. A total of 12 slides were produced from 6 animals.

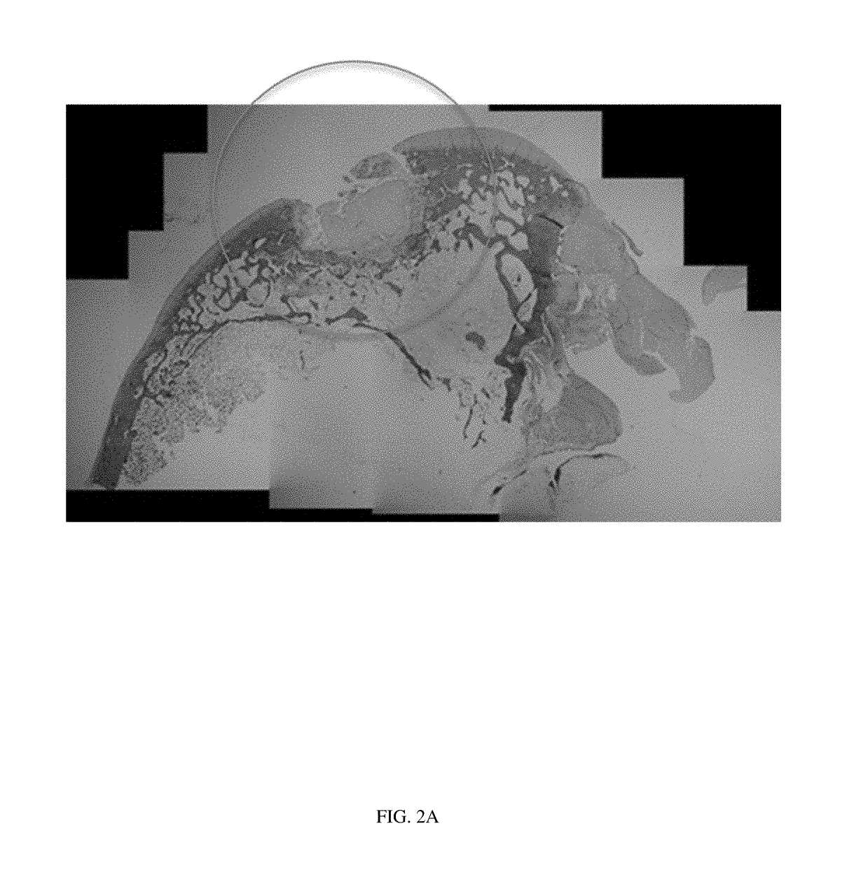

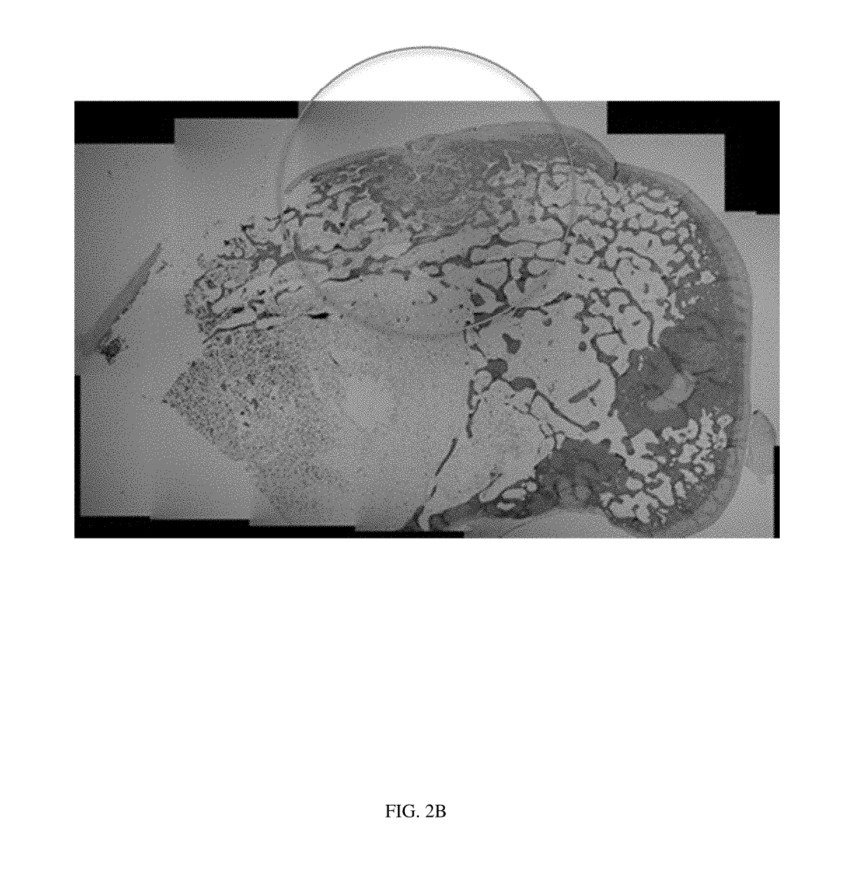

High-resolution digital images were acquired by field for the all surgical site slides using a Nikon E800 microscope (AG Heinze, Lake Forest, Calif.), Spot digital camera (Diagnostic Instruments, Sterling, Heights, Mich.), a Pentium IBM-based computer with expanded memory capabilities (Dell Computer Corp., Round Rock, Tex.). See FIGS. 2A and 2B.

### Results

Results from a pilot study in 6 rabbits with a 6 week in vivo follow-up. This early timepoint was chosen to show differences in the healing.

Table 1 below indicates the scores of the blinded DVM scoring of histology sections.

TABLE 1
BLINDED DVM SCORING

| Animal ID | Type of Repair | Cartilage Defect Repair Scores |
|---|---|---|
| 215L | Nanofiber scaffold | 0 |
| | | 0 |
| 215R | Cartiform ® | 3 |
| | | 3 |
| 217L | Cartiform ® | 0 |
| | | 0 |
| 217R | Nanofiber scaffold | 1 |
| | | 2 |
| 218L | Nanofiber scaffold | 2 |
| | | 2 |
| 218R | Cartiform ® | 3 |
| | | 3 |
| 219L | Cartiform ® | 0 |
| | | 0 |
| 219R | Nanofiber scaffold | 4 |
| | | 4 |
| 220L | Nanofiber scaffold | 1 |
| | | 1 |
| 220R | Cartiform ® | 1 |
| | | 1 |
| 221L | Cartiform ® | 1 |
| | | 1 |
| 221R | Nanofiber scaffold | 1 |
| | | 2 |

| | |
|---|---|
| Cartiform ® Avg. | 1.33 |
| Nanofiber scaffold avg. | 1.67 |

Animal 215 L had a very large defect created by accident during implantation and should be removed from the analyses which would increase the Nanofiber Scaffold score to 2.00.

FIG. 2A shows the histological sample of the femur repaired by Cartiform. Centrally, this defect is filled with a large devitalized fragment of hyaline-like cartilage (allograft). Circumferentially surrounding this allograft, lining the margin of the defect, and extending into and filling the adjacent medullary spaces is moderate amounts of dense fibrous connective tissue. There is no histological evidence of re-establishment of an articular cartilage surface.

FIG. 2B shows the histological sample of the rabbit femur repaired using a nanofiber scaffold per an embodiments of the disclosure herein. As shown by the figure, the defect is filled with tissue and appears well-integrated into the surrounding host bone. Completely filling the defect is an approximately 50/50 mixture composed of trabeculae of new woven bone or hyaline-like cartilage.

Diffusely, the superficial surface of the defect is completely filled with dense hyaline cartilage. This cartilage is disorganized and lacks normal hyaline-structure of chondrocytes but is re-establishing the normal articular surface.

Although embodiments herein have been described in considerable detail with reference to certain preferred embodiments thereof, other versions are possible. Therefore, the spirit and scope of the appended claims should not be limited to the description and the preferred versions contained within this specification.

1. A method of treating hyaline cartilage damage in a subject in need thereof, the method comprising placing at least one electrospun polymer fiber in physical communication with a damaged hyaline cartilage of the subject.

2. The method of claim 1, wherein the at least one electrospun polymer fiber comprises a polymer selected from the group consisting of polyethylene terephthalate, polyurethane, polyethylene, polyethylene oxide, polyester, polymethylmethacrylate, polyacrylonitrile, silicone, polycarbonate, polyether ketone ketone, polyether ether ketone, polyether imide, polyamide, polystyrene, polyether sulfone, polysulfone, polyvinyl acetate, polytetrafluoroethylene, polyvinylidene fluoride, polycaprolactone, polylactic acid, polyglycolic acid, polylactide-co-glycolide, polylactide-co-caprolactone, polydioxanone, Poly(3-hydroxybutyrate-co-3-hydroxyvalerate), trimethylene carbonate, polydiols, polyesters, collagen, gelatin, fibrin, fibronectin, albumin, hyaluronic acid, elastin, chitosan, alginate, silk, copolymers thereof, and combinations thereof.

3. The method of claim 1, wherein the at least one electrospun polymer fiber comprises a plurality of electrospun polymer fibers having an orientation relative to one another that is random, substantially parallel, or a combination thereof.

4. The method of claim 1, wherein the at least one electrospun polymer fiber comprises a plug.

5. The method of claim 4, wherein the plug has a length from about 1 mm to about 100 mm, and wherein the patch has a width from about 1 mm to about 100 mm.

6. The method of claim 4, wherein the patch has a thickness from about 100 μm to about 10,000 m.

7. The method of claim 4, wherein the patch comprises pores having a diameter from about 0.25 μm to about 50 μm.

8. The method of claim 4, wherein the patch has a Young's modulus from about 0.5 MPa to about 1,000 MPa.

9. The method of claim 4, wherein the patch is configured to facilitate the migration of cells.

10. The method of claim 4, wherein the patch further comprises a component selected from the group consisting of tricalcium phosphate, hydroxyapatite, bioglass, mesenchymal stem cells, tenocytes, fibroblasts, osteoblasts, platelet-rich plasma, stromal vascular fraction, bursa cells, amnion, growth factors, and combinations thereof.

11. The method of claim 1, further comprising securing the at least one electrospun polymer fiber in physical communication with the damaged hyaline cartilage using at least one suture.

12. The method of claim 1, further comprising securing the at least one electrospun polymer fiber in physical communication with the damaged hyaline cartilage using a biological adhesive.

13. The method of claim 12, wherein the biological adhesive is selected from the group consisting of a fibrin sealant, an autologous fibrin sealant, a gelatin-resorcinol aldehyde, a protein-aldehyde system, a collagen-based adhesive, a polysaccharide-based adhesive, a mussel adhesive protein, variants thereof, and combinations thereof.

14. The method of claim 1, wherein method does not include seeding the at least one electrospun polymer fiber with cells prior to placing the at least one electrospun polymer fiber in physical communication with the damaged hyaline cartilage.

15. The method of claim 1, wherein the method does not include administering a biologic.

16. A method of treating arthritis in a subject in need thereof, the method comprising placing at least one electrospun polymer fiber in physical communication with a hyaline cartilage of the subject.

17. The method of claim 16, wherein the arthritis is selected from the group consisting of osteoarthritis and rheumatoid arthritis.

18. The method of claim 16, wherein the at least one electrospun polymer fiber comprises a polymer selected from the group consisting of polyethylene terephthalate, polyurethane, polyethylene, polyethylene oxide, polyester, polymethylmethacrylate, polyacrylonitrile, silicone, polycarbonate, polyether ketone ketone, polyether ether ketone, polyether imide, polyamide, polystyrene, polyether sulfone, polysulfone, polyvinyl acetate, polytetrafluoroethylene, polyvinylidene fluoride, polycaprolactone, polylactic acid, polyglycolic acid, polylactide-co-caprolactone, polydioxanone, Poly(3-hydroxybutyrate-co-3-hydroxyvalerate), trimethylene carbonate, polydiols, polyesters, collagen, gelatin, fibrin, fibronectin, albumin, hyaluronic acid, elastin, chitosan, alginate, silk, copolymers thereof, and combinations thereof.

19. The method of claim 16, wherein the at least one electrospun polymer fiber comprises a patch.

20. The method of claim 19, wherein the patch is configured to facilitate the migration of cells.
